(19) United States
(12) Patent Application Publication
(10) Pub. No.: US 2011/0189164 A1
(43) Pub. Date: Aug. 4, 2011

(54) Peptide Stabilizer Compounds and Screening Method

(75) Inventors: Eldridge William, Essex (GB); Fitzgerald Kevin, Essex (GB); Cooley Neil, Cambridge (GB); McGregor Duncan, Suffolk (GB)

(73) Assignee: Isogenica Ltd, Cambridgeshire (GB)

(21) Appl. No.: 11/908,790

(22) PCT Filed: Mar. 16, 2006
(86) PCT No.: PCT/GB2006/000967

§ 371 Date: Dec. 9, 2008

(30) Foreign Application Priority Data
Mar. 16, 2005 (GB) 0505420.0

(51) Int. Cl. A61K 39/395; C12Q 1/68; C07K 7/08; C12N 9/96; C07K 14/00; C07K 16/00; A61K 38/00; A61K 38/54

(52) U.S. Cl. 424/130.1; 435/6.1; 530/327; 435/188; 530/399; 530/387.1; 514/1.1; 424/94.3

(57) ABSTRACT

Peptide stabilizer compounds are provided that in combination with a biologically active peptide, can increase the protease elimination half-time of the biologically active peptide in vivo. The peptide stabilizer compounds are preferably in the form of peptide sequences that confer resistance to proteolysis upon conjugated biologically active peptides. Also provided is a method for the selection of novel proteolysis resistant compounds from in vitro generated libraries, and pharmaceutical compositions comprising the stabilizer compounds identified thereby.

### FIELD

This invention relates to novel compounds termed peptide stabilizers that are resistant to proteolysis. In particular aspects, the invention relates to compositions comprising a hybrid molecule comprising a peptide stabilizer moiety and a bioactive moiety such as a biologically active molecule. The active moiety may comprise a molecule useful for diagnostic or therapeutic purposes.

### BACKGROUND

Bioactive peptides are small peptides that elicit a biological activity. Over 500 of these peptides which average 20 amino acids in size have been identified and characterized. They have been isolated from a variety of natural or non-natural systems, exhibit a wide range of actions, and have been utilized as therapeutic agents in the field of medicine and as diagnostic tools in both basic and applied research. Where the mode of action of these peptides has been determined, it has been found to be due to the interaction of the bioactive peptide with a specific protein target. In most of the cases, the bioactive peptide acts by binding to and inactivating its protein target with extremely high specificities. Recently, there has been an increasing interest in employing synthetically derived bioactive peptides as novel pharmaceutical agents due to the impressive ability of the naturally occurring peptides to bind to and modulate the activity of specific protein targets.

Novel bioactive peptides have been engineered previously through the use of two different in vitro approaches. The first approach produces candidate peptides by chemically synthesizing a randomized library of 6-10 amino acid peptides (J. Eichler et al., Med. Res. Rev. 15:481-496 (1995); K. Lam, Anticancer Drug Des. 12:145-167 (1996); M. Lebl et al., Methods Enzymol. 289:336-392 (1997)). In the second approach, candidate peptides are synthesized by cloning a randomized oligonucleotide library into a Ff filamentous phage gene, which allows peptides that are much larger in size to be expressed on the surface of the bacteriophage (H. Lowman, Ann. Rev. Biophys. Biomol. Struct. 26:401-424 (1997); G. Smith et al., et al. Meth. Enz. 217:228-257 (1993)). To date, randomized peptide libraries up to 38 amino acids in length have been made, and longer peptides are likely achievable using this system. The peptide libraries that are produced using either of these strategies are then typically mixed with a pre-selected matrix-bound protein target. Peptides that bind are eluted, and their sequences are determined. From this information new peptides are synthesized and their biological properties are determined.

Phage-display provides a means for generating constrained and unconstrained peptide libraries (Devlin et al., (1990) Science 249:404-406; Cwirla et al., (1990) Proc. Natl. Acad. Sci. USA 87:6378-6382; Lowman (1997) Ann. Rev. Biophys. Biomol. Struct. 26:401-424). These libraries can be used to identify and select synthetic peptides that can bind a predetermined target molecule. Both phage display and chemically synthesized libraries are limited by the size of libraries that can be generated to within the 10⁶-10⁹ range. This limitation has resulted in the isolation of peptides of relatively low affinity without time consuming subsequent maturation. This limitation has led to the development of techniques for in vitro generation of libraries including mRNA display (Roberts, & Szostak, Proc. Natl. Acad. Sci. USA 94, 12297-12302. (1997)), ribosome display (Mattheakis et al Proc. Natl. Acad. Sci. USA 91, 9022-9026 (1994)) and CIS display (Odegrip et al Proc. Natl. Acad. Sci USA 101 2806-2810 (2004)) amongst others. These libraries are superior to phage display libraries in that the size of libraries generated by such methods is 2-3 orders of magnitudes larger than is possible with phage display. However, these have proved no more successful than phage display in isolating peptides with a combination of protease resistant and bioactive properties.

Although these in vitro approaches show promise, the use of synthetically derived peptides has not yet become a mainstay in the pharmaceutical industry. The primary obstacle remaining is that of peptide instability within the biological system of interest as evidenced by the unwanted degradation of potential peptide drugs by proteases and/or peptidases in the host. Approaches used to address the problem of peptide degradation have included the use of D-amino acids or modified amino acids as opposed to the naturally occurring L-amino acids (e.g., J. Eichler et al., Med Res Rev. 15:481-496 (1995); L. Sanders, Eur. J. Drug Metabol. Pharmacokinetics 15: 95-102 (1990)), the use of cyclized peptides (e.g., R. Egleton, et al., Peptides 18: 1431-1439 (1997)), the isolation of protease resistant viral particles (WO9958655) and the development of enhanced delivery systems that prevent degradation of a peptide before it reaches its target in a patient (e.g., L. Wearley, Crit. Rev. Ther. Drug Carrier Syst. 8: 331-394 (1991); L. Sanders, Eur. J. Drug Metabol. Pharmacokinetics 15: 95-102 (1990)). Although these approaches for stabilizing peptides and thereby preventing their unwanted degradation in the biosystem of choice (e.g., a patient) are promising, there remains no way to routinely and reliably stabilize peptide drugs and drug candidates. Moreover, many of the existing stabilization and delivery methods cannot be directly utilized in the screening and development of novel useful bioactive peptides. Hence, methods for stabilizing biologically active peptides and also methods for identifying novel peptide stabilizer compounds would be desirable. Such methods would provide a much-needed advance in the field of peptide drug development.

These and other uses, features and advantages of the invention should be apparent to those skilled in the art from the teachings provided herein.

### SUMMARY OF THE INVENTION

In a first aspect, the present invention provides a method for the selection of novel proteolysis resistant compounds (referred to as peptide stabilizers) from in vitro generated libraries comprising a plurality of peptides, comprising the steps of:
a) expressing a plurality of nucleic acid constructs, wherein each nucleic acid construct encodes a hybrid peptide that comprises:
   i) a bioactive moiety; and
   ii) a putative peptide stabilizer moiety;
b) exposing the expressed hybrid peptides to one or more proteases;
c) exposing the expressed hybrid peptides to a target molecule which is capable of interacting with the bioactive moiety in a detectable manner; and
d) if a detectable interaction occurs between a target molecule and the bioactive moiety of one or more of the expressed hybrid peptides, identifying said one or more expressed hybrid peptides as comprising a peptide stabilizer compound.

The peptide stabilizer libraries of the present invention are composed of, for example, peptides or peptide derivatives such as peptide mimetics and peptide analogs composed of naturally occurring or non-natural amino acids. According to the invention, the peptide stabilizers isolated by the invention are non-naturally occurring amino acid sequences that are resistant to proteolysis by enzymes such as trypsin or thrombin by way of example. Preferably the peptide stabilizer is a non-naturally occurring amino acid sequence of between about 6 and about 30 amino acid residues, more preferably between about 7 and about 25 amino acid residues, and most preferably between about 8 and 20 amino acid residues.

Optionally the invention is directed to selection of combinations of a peptide stabilizer with a bioactive peptide to form a hybrid molecule that comprises a peptide stabilizer moiety and a bioactive moiety. The protease resistance of the peptide stabilizer moiety increases the protease elimination time half time (or half life) of the hybrid molecule.

Such compounds preferably are selected by the invention to be resistant to proteolysis by a desired protease or peptidase for protease elimination half times of greater than 30 minutes, preferably greater than 3 hours at the desired enzymes optimal activity conditions, and preferably is resistant to proteolysis by incidental enzymes. Specific examples of such compounds include linear or cyclic peptides, preferably between about 8 and 20 amino acid residues in length, and combinations thereof, optionally modified at the N-terminus or C-terminus or both, as well as their salts and derivatives, functional analogues thereof and extended peptide chains carrying amino acids or polypeptides at the termini of the sequences.

According to one embodiment of the invention, libraries of in vitro generated nucleic acid-peptide stabilizers are generated by an appropriate method, fused to a bioactive molecule and selected for binding to the bioactive molecule target in the presence of a desired peptidase or protease. Library members capable of binding to the target and resistant to the desired protease or peptidase are then recovered and characterised. According to the invention the desired peptidase or protease is added to the library before, during or after binding to the bioactive molecule target. Optionally the invention provides peptide stabilizer moieties that can be linked to different bioactive moieties to increase the elimination time half-life of each of the hybrid molecules. The invention also provides for the selection of a hybrid ligand with two or more peptide stabilizer moieties and one bioactive moiety or any other combinations that can be envisaged by one skilled in the art.

According to the invention a hybrid molecule can be selected in which the bioactive moiety has the peptide stabilizer moiety incorporated within the bioactive moiety sequence.

Thus, in a specific embodiment of the invention, a proteolysis resistant hybrid molecule can be selected from a library of sequences derived from a bioactive moiety peptide sequence by the following method:
a) expressing a plurality of nucleic acid constructs, wherein each nucleic acid construct encodes a hybrid peptide derived from the bioactive peptide sequence that comprises:
   i) a bioactive moiety; and
   ii) a putative peptide stabilizer moiety;
b) exposing the expressed hybrid peptides to one or more proteases;
c) exposing the expressed hybrid peptides to a target molecule which is capable of interacting with the bioactive moiety in a detectable manner; and
d) if a detectable interaction occurs between a target molecule and the bioactive moiety of one or more of the expressed hybrid peptides, identifying said one or more expressed hybrid peptides as comprising a peptide stabilizer compound.

The bioactive peptides of the invention include any compound useful as a therapeutic or diagnostic agent that can be incorporated into an in vitro synthesized nucleic acid encoded library. Non-limiting examples of bioactive peptides include enzymes, hormones, cytokines, antibodies or antibody fragments, peptide fragments recognised by antibodies, analgesics, antipyretics, anti-inflammatory agents, antibiotics, antiviral agents, anti-fungal drugs, cardiovascular drugs, drugs that affect renal function and electrolyte metabolism, drugs that act on the central nervous system and chemotherapeutic drugs, to name but a few.

According to the invention, the hybrid molecules comprising a peptide stabilizer moiety and a bioactive peptide moiety have improved pharmacokinetic or pharmacodynamic properties as compared to the same bioactive peptide comprising the active moiety but lacking the peptide stabilizer moiety. The improved pharmacokinetic or pharmacodynamic properties of the hybrids thereby provide for low-dose pharmaceutical formulations and novel pharmaceutical compositions. The invention provides for methods of using the novel compositions including the therapeutic or diagnostic use of the hybrid molecules.

The invention is directed to combinations of peptide stabilizers, which increase protease elimination half times with bioactive peptides that have relatively short protease elimination half-times. The combinations are selected with various objectives in mind, including improving the therapeutic or diagnostic efficacy of the bioactive peptide when the invention involves in vivo use of the bioactive peptide, for example, increasing the protease elimination half-time of the bioactive compound. Genetically fusing or chemically linking (i.e., “conjugating”) the peptide stabilizer to a bioactive peptide provides compositions with increased protease elimination half-times.

Accordingly, a further aspect of the invention provides a pharmaceutical composition comprising at least one peptide stabilizer compound identified according to the methods described above, a bioactive molecule and a suitable carrier. Pharmaceutical compositions of the invention are formulated to conform with regulatory standards and can be administered orally, intra-venously, intra-nasally, topically, or via other standard routes. The pharmaceutical compositions may be in the form of tablets, pills, lotions, gels, liquids, powders, suppositories, suspensions, sprays, liposomes, microparticles or other suitable formulations known in the art.

A further aspect of the invention provides a method for imparting proteolysis resistance to a bioactive peptide molecule comprising, linking a peptide stabilizer compound, identified by the method described above, to the bioactive peptide molecule.

All references cited herein are incorporated by reference in their entirety. Unless otherwise defined, all technical and scientific terms used herein have the same meaning as commonly understood by one of ordinary skill in the art to which this invention belongs.

### DESCRIPTION OF FIGURES

FIG. 1 shows a diagrammatic representation of a construct for use in identifying peptide stabilizer sequences in the thrombin resistance peptide library of Example 1. X-X-X is a representation of the random 12mer peptide library separating the FLAG epitope and thrombin cleavage site. Arrows indicate potential thrombin (Pro-Arg) and trypsin cleavage sites (Lys or Arg).

FIG. 2 shows thrombin resistant peptides after five rounds of selection. The values represent ((OD450 nm reading after incubation with thrombin)/(OD450 nm reading after incubation without thrombin)) expressed as a percentage. 100% indicates complete protection from thrombin cleavage. Control bars are the observed resistance for the peptide shown in SEQ ID NO: 002.

FIG. 3 shows trypsin resistant peptides. A. Unselected library peptides incubated with trypsin. B. Unselected library peptides incubated without trypsin. C. Round five thrombin selection peptides incubated with trypsin. D. Round five thrombin selection peptides incubated without trypsin.

FIG. 4 shows a diagrammatic representation of a construct for use in identifying peptide stabilizer sequences in the trypsin/chymotrypsin resistance peptide library of Example 2.

FIG. 5 shows a ethidium bromide stained agarose gel photograph of PCR recoveries from rounds 1-4 of Example 2, selected on anti-FLAG antibody with chymotrypsin/trypsin treatment (CT) or no protease treatment (NP).

FIG. 6 shows the nucleic acid sequences of the PCR primers that are used in library construction according to Example 2.

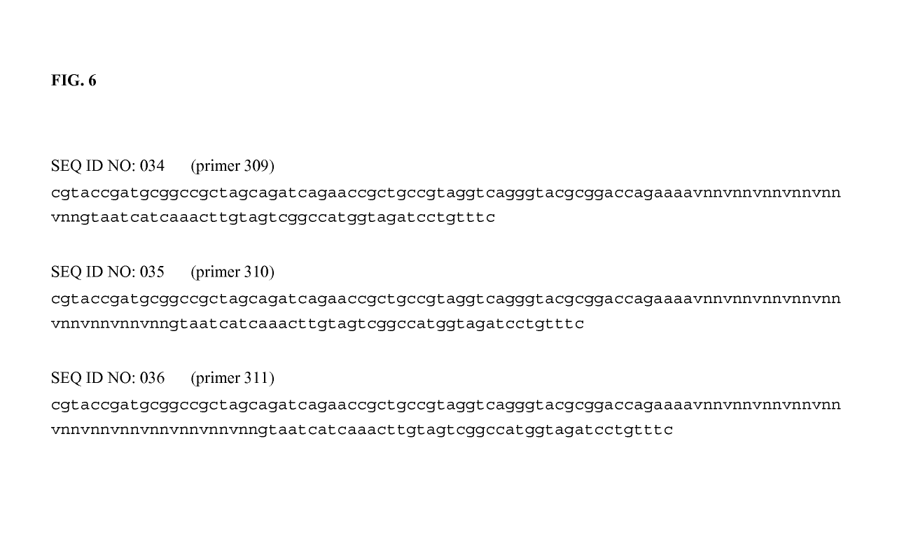

### DETAILED DESCRIPTION

The terms “peptide”, “peptide stabilizer”, “bioactive peptide” and “hybrid molecule” as used herein refers to a plurality of amino acids joined together in a linear chain. Accordingly, the terms “peptide”, “peptide stabilizer”, “bioactive peptide” and “hybrid molecule” as used herein includes a dipeptide, tripeptide, oligopeptide and polypeptide. A dipeptide contains two amino acids; a tripeptide contains three amino acids; and the term oligopeptide is typically used to describe peptides having between 2 and about 50 or more amino acids. Peptides larger than about 50 are often referred to as polypeptides or proteins. For purposes of the present invention, the terms “peptide”, “peptide stabilizer”, “bioactive peptide” and “hybrid molecule” are not limited to any particular number of amino acids. Preferably, however, they contain about 2 to about 50 amino acids, more preferably about 2 to about 40 amino acids, most preferably about 2 to about 20 amino acids.

“Peptide stabilizers”, “bioactive peptides” and “hybrid molecules” as used herein are amino acid sequences as described above which may contain naturally as well as non-naturally occurring amino acid residues. Therefore, so-called “peptide mimetics” and “peptide analogs” which may include non-amino acid chemical structures that mimic the structure of a particular amino acid or peptide may be peptide stabilizers within the context of the invention. Such mimetics or analogues are characterized generally as exhibiting similar physical characteristics such as size, charge or hydrophobicity present in the appropriate spacial orientation as found in their peptide counterparts. A specific example of a peptide mimetic compound is a compound in which the amide bond between one or more of the amino acids is replaced by, for example, a carbon-carbon bond or other bond as is well known in the art (see, for example Sawyer, in Peptide Based Drug Design pp. 378-422 (ACS, Washington D.C. 1995)).

Therefore, the term “amino acid” within the scope of the present invention is used in its broadest sense and is meant to include naturally occurring L .alpha.-amino acids or residues. The commonly used one and three letter abbreviations for naturally occurring amino acids are used herein (Lehninger, A. L., Biochemistry, 2d ed., pp. 71-92, (1975), Worth Publishers, New York). The correspondence between the standard single letter codes and the standard three letter codes is well known to one skilled in the art, and is reproduced here: A=Ala; C=Cys; D=Asp; E=Glu; F Phe; G=Gly; H His; I=Ile; K=Lys; L=Leu; M=Met; N=Asn; P=Pro; Q=Gln; R=Arg; S=Ser; T=Thr, V=Val; W=Trp; Y=Tyr. The term includes D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio The Peptides: Analysis, Synthesis, Biology, Gross and Meiehofer, eds., Vol. 5 p. 341, Academic Press, Inc., N.Y. 1983, which is incorporated herein by reference.

Peptide stabilizers, when used within the context of the present invention, may be “conjugated” to a bioactive peptide. The term “conjugated” is used in its broadest sense to encompass all methods of attachment or joining that are known in the art. For example, the peptide stabilizer will be an amino acid extension of the C- or N-terminus of the bioactive peptide. In addition, a short amino acid linker sequence may lie between the bioactive peptide and the peptide stabilizer. In this scenario, the peptide stabilizer, optional linker and bioactive peptide will be coded for by a nucleic acid comprising a sequence encoding bioactive peptide operably linked to (in the sense that the nucleic acid sequences are contiguous and in reading frame) an optional linker sequence encoding a short polypeptide, and a sequence encoding the peptide stabilizer. In this typical scenario, the peptide stabilizer is considered to be “conjugated” to the bioactive peptide optionally via a linker sequence. According to the invention the peptide stabilizer amino acid sequence may interrupt or replace a section of the bioactive peptide amino acid sequence, provided, of course, that the insertion of the peptide stabilizer amino acid sequence does not interfere with the function of the bioactive peptide. Optionally the invention provides for a “conjugate” that can be coded for by a nucleic acid comprising a sequence encoding bioactive peptide interrupted by and operably linked to a sequence encoding the peptide stabilizer. The invention further provides for hybrid molecules where the peptide will be linked, e.g. by chemical conjugation to the bioactive peptide or other therapeutic compound optionally via a linker sequence. Typically, the peptide stabilizer will be linked to the bioactive peptide via a side chain of an amino acid somewhere in the middle of the bioactive peptide that doesn't interfere with the bioactive peptide's activity. Here again, the peptide is considered to be “conjugated” to the bioactive peptide.

“Protease elimination half-time/half-life” is used as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination by proteolytic activity. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2}, the time required for 50% completion of the process. The units of these two constants are time⁻¹ and time, respectively. A first-order rate constant and the half-time of the reaction are simply related (k·times t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination by protease or peptidase activity can be accurately determined from such a graph.

The present invention represents a significant advance in the art of peptide drug development by allowing concurrent screening of in vitro generated libraries for peptide bioactivity and stability.

In vitro generated nucleic acid libraries encoding a plurality of peptides are synthesised and selected for binding to a target in the presence of one or more desired proteases or peptidases. Library members incapable of binding to the target or, more importantly, incapable of binding to the target in the presence of the desired protease or peptidase are removed by washing or other methods known to those skilled in the art. Library members encoding a peptide stabilizer moiety and a bioactive peptide moiety will remain bound to the target. These target bound hybrid molecules encoding peptide stabilizer and bioactive peptide library members are then recovered and individually characterised by sequencing the associated nucleic acid, and expressing or synthesising the encoded hybrid molecule to confirm bioactive peptide target binding and desired protease or peptidase resistance in the peptide stabilizer moiety. Optionally, the invention provides hybrid molecules that surprisingly still possesses one or more known cleavage sites of the desired protease or peptidase but is rendered resistant to cleavage by that protease or peptidase by the peptide stabilizer moiety.

According to the invention in vitro generated nucleic acid libraries encoding a plurality of peptides and a separate bioactive peptide are synthesised in such a manner that protease cleavage of the bioactive peptide will disrupt the linkage between encoding nucleic acid and the bioactive peptide and library peptide. The library is selected for binding to the bioactive peptide target in the presence of one or more desired proteases or peptidases. Library members incapable of binding to the target or, more importantly, incapable of protecting the bioactive peptide from cleavage in the presence of the desired protease or peptidase are removed by washing or other methods known to those skilled in the art. These target bound hybrid molecules encoding peptide stabilizer and bioactive peptide library members are then recovered and individually characterised by sequencing the associated nucleic acid, and expressing or synthesising the encoded hybrid molecule to confirm bioactive peptide target binding and desired protease or peptidase resistance in the peptide stabilizer moiety. According to the invention certain of the peptide stabilizer moieties isolated are anticipated to protect other bioactive peptides not used in the selection. Optionally, certain of the peptide stabilizer moieties isolated according to the invention are anticipated to protect this or other bioactive peptides against peptidases and proteases not used in the selection as surprisingly evidenced from certain of the thrombin resistant peptide stabilizer moieties described in example 1 being additionally resistant to trypsin. Certain of the peptide stabilizer moieties described in example 1 have utility in preventing proteolytic degradation of bioactive peptides. By way of example the peptide stabilizer moieties of the invention can be conjugated to the bioactive peptide parathyroid hormone to produce a hybrid molecule of the invention with increased protease resistance. Such hybrid molecules have utility as improved pharmaceutical compounds for the treatment of osteoporosis resulting from increased protease resistance and prolonged bioactivity.

Optionally, certain of the nucleic acid pools encoding peptide stabilizer moieties isolated according to the invention, such as those described in example 1, have utility in preventing proteolytic degradation of bioactive peptides. By way of example the nucleic acid pool enriched for peptide stabilizer moieties of the invention from the fourth or fifth rounds of selection described in example 1 can be fused to the nucleic acid encoding any bioactive peptide to produce a hybrid molecule of the invention. Such pools can be further screened by means of the invention, or any method known to those skilled in the art, for peptide stabilizer moieties most suitable for the desired bioactive peptide.

The invention provides for a biased in vitro nucleic acid-peptide library based on an existing bioactive peptide sequence so that at any one position in the bioactive peptide a majority of the library will code for the actual amino acid present in the bioactive peptide. Many methods for generating such libraries with chemical DNA oligonucleotide synthesis and PCR are known to one skilled in the art such as biasing nucleotide usage (Wolf & Kim, Protein Sci. 8: 680-688 (1999)) using trinucleotide mutagenesis (Sondek & Shortie, Proc. Natl. Acad. Sci. USA 89:3581-3585) and these can be applied to all in vitro display library methods. The library is selected for binding to the bioactive peptide target in the presence of one or more desired proteases or peptidases. Library members incapable of binding to the target or, more importantly, incapable of protecting the bioactive peptide from cleavage in the presence of the desired protease or peptidase are removed by washing or other methods known to those skilled in the art. These target bound hybrid molecules encoding peptide stabilizer and bioactive peptide library members are then recovered and individually characterised by sequencing the associated nucleic acid, and expressing or synthesising the encoded hybrid molecule to confirm bioactive peptide target binding and desired protease or peptidase resistance in the peptide stabilizer moiety. According to the invention the peptide stabilizer moiety is found either within the bioactive peptide moiety or conjugated thereto. Optionally, two or more peptide stabilizer moieties may be encoded within the bioactive peptide moiety. Hence, the invention provides peptide stabilizer moieties that protect or enhance the biological activity of the bioactive moiety without altering the size of the hybrid molecule from the size of the original bioactive moiety. Hybrid molecules of the invention derived from the bioactive moiety have utility as improved therapeutic agents through prolonged or enhanced biological activity in a patient. Said prolonged or enhanced biological activity being derived from the peptide stabilizer moiety.

Optionally the invention provides for protease resistant hybrid molecules isolated herein, suitable for oral administration as a result of the peptide stabilizer moiety incorporated into the hybrid molecule. Preferably the orally administered hybrid molecule is composed solely of naturally occurring L-amino acids. Said hybrid molecules may be optionally modified at the N- and C-termini by amidation or carboxylation or other such method known to those skilled in the art. By way of example not exclusion, bioactive peptides such as those described in WO-A-0215923 (for use in treating atherosclerosis) composed solely of L-amino acids when incorporated into a hybrid molecule of the current invention would be rendered suitable for oral administration. Such hybrid molecules of the invention derived from a bioactive moiety of WO-A-0215923 would have utility as improved therapeutic agents through prolonged biological activity in a patient. Said prolonged biological activity being derived from the peptide stabilizer moiety of the current invention.

Thus, the identified hybrid molecules of the present invention can have utility as pharmaceutical compounds for the prevention of heart disease and can be administered intravenously, by intramuscular injection, or more preferably, orally.

It is also possible to use a variant of the “peptide stabilizer” described herein. A number of variants are possible. A variant can be prepared and then tested for protease resistance, e.g., using a binding assay such as ELISA. One type of variant is a truncation of the “peptide stabilizer” described herein. In this example, the variant is prepared by removing one or more amino acid residues of the “peptide stabilizer” from the N or C terminus. In some cases, a series of such variants is prepared and tested. Information from testing the series is used to determine a region of the “peptide stabilizer” that is essential for protease resistance. A series of internal deletions or insertions can be similarly constructed and tested.

Another type of variant is a substitution. In one example, the “peptide stabilizer” is subjected to alanine scanning to identify residues that contribute to stabilising activity. In another example, a library of substitutions at one or more positions is constructed. The library may be unbiased or, particularly if multiple positions are varied, biased towards an original residue. In some cases, the substations are limited to conservative substitutions.

A related type of variant is a “peptide stabilizer” that includes one or more non-naturally occurring amino acids. Such variant ligands can be produced by chemical synthesis. One or more positions can be substituted with a non-naturally occurring amino acid. In some cases, the substituted amino acid may be chemically related to the original naturally occurring residue (e.g., aliphatic, charged, basic, acidic, aromatic, hydrophilic) or an isostere of the original residue.

It may also be possible to include non-peptide linkages and other chemical modification. For example, part or all of the “peptide stabilizer” may be synthesized as a peptidomimetic, e.g., a peptoid (see, e.g., Simon et al. (1992) Proc. Natl. Acad. Sci. USA 89:9367-71 and Horwell (1995) Trends Biotechnol. 13:132-4). A peptide may include one or more (e.g., all) non-hydrolyzable bonds. Many non-hydrolyzable peptide bonds are known in the art, along with procedures for synthesis of peptides containing such bonds. Exemplary non-hydrolyzable bonds include —[CH.sub.2NH]— reduced amide peptide bonds, —[COCH₂]— ketomethylene peptide bonds, —[CH(CN)NH]— (cyanomethylene)amino peptide bonds, —[CH₂CH(OH)]— hydroxyethylene peptide bonds, —[CH₂O]— peptide bonds, and —[CH₂S]— thiomethylene peptide bonds (see e.g., U.S. Pat. No. 6,172,043).

### EXAMPLES

The following procedures used by the present applicant are described in Sambrook, J., et al., 1989 supra.: analysis of restriction enzyme digestion products on agarose gels and preparation of phosphate buffered saline.

General purpose reagents were purchased from SIGMA-Aldrich Ltd (Poole, Dorset, U.K.). Oligonucleotides were obtained from Eurogentec Ltd (Southampton, U.K.). Amino acids, and S30 extracts were obtained from Promega Ltd (Southampton, Hampshire, U.K.). Proteases were purchased from SIGMA-Aldrich (Poole, Dorset, U.K.) or Novagen (Nottingham, UK). Vent and Taq DNA polymerases were obtained from New England Biolabs (Cambridgeshire, U.K.). Anti-human IgK antibodies from Immunologicals Direct Ltd (Oxfordshire, U.K.) and M2 anti-FLAG polyclonal from SIGMA-Aldrich Ltd (Poole, Dorset, U.K.).

### Example 1

### Identification of Peptide Stabilizers that Protect Against Cleavage by Thrombin and Trypsin

To exemplify the invention thrombin protease resistant peptides were selected from a N-terminal cis display library genetically fused to RepA, under the control of a tac promoter constructed to contain:

a. a FLAG peptide moiety capable of binding to an anti-FLAG antibody (bioactive peptide 1), and,

b. a twelve amino acid random library from which thrombin resistant peptide stabilizer moieties may be selected and,

c. a four amino acid moiety capable of being cleaved by thrombin protease (bioactive peptide 2)

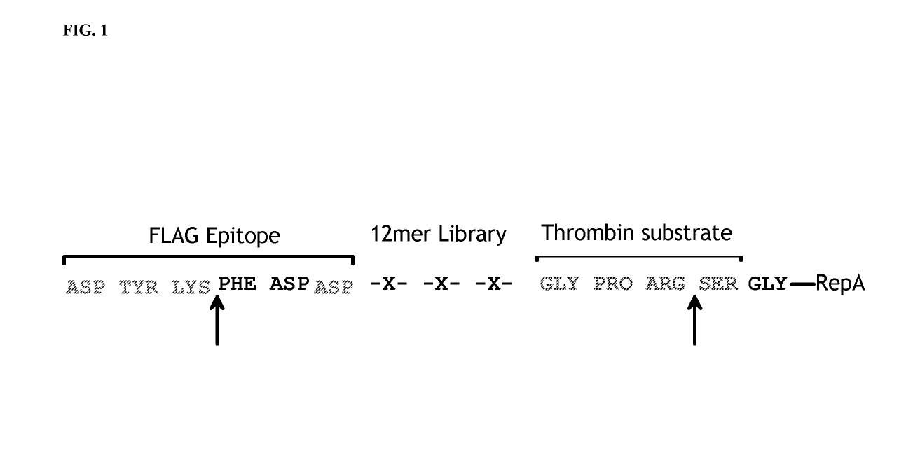

as shown in FIG. 1. Library construction and in vitro transcription and translation were carried out as described by Odegrip et al (Proc. Natl. Acad. Sci USA 101 2806-2810 (2004). The tac-FLAG-NNB-Thrombin cleavage site-RepA-CIS-ori PCR construct was prepared by appending a FLAG epitope and a 12-mer NNB library (where N is any nucleotide and B is either C, T or G), followed by a thrombin cleavage sequence (encoding GPRS—where “R”=P1) to the tac promoter by PCR with primer TRL (SEQ ID NO: 001) and then ligating it to the RepA-CIS-ori region followed by PCR amplification. In vitro transcription and translation was performed in an E. coli S-30 lysate system (30) for up to 30 minutes at 30° C. and then diluted with blocking buffer (2% BSA, 0.1 mg/ml herring sperm DNA, in PBS). Typically, 2 ng of linear DNA was added per 50 μl of S-30 lysate. Biotinylated anti-FLAG (M2) (SIGMA) antibody was added to a final concentration of 1 μg/ml. Human Thrombin (SIGMA) was added to a final concentration of 0.1 unit/ml. Solutions were incubated for 2 h at 25° C. in rounds 1, 2 and 3 of selection procedure and for 2 h at 37° C. in rounds 4 and 5. Streptavidin-coated Dynal M280 paramagnetic beads were added at 50 μl/ml of solution and incubated for 10 minutes at room temperature before extensive washing with PBS/0.1%-Tween-20. DNA was eluted, purified and the N-terminal library region amplified and reassembled with the RepA-CIS-ori, as described above, to produce input DNA for the next round of selection. In order for selection to work the bioactive peptide thrombin substrate sequence must be protected from cleavage by the peptide stabilizers encoded in the random library region.

Recovered DNA from the fifth round of selection was amplified using PCR, purified and digested with NotI and NcoI. The DNA was then ligated into a similarly digested M13 gpIII phagemid vector and transformed into E. coli XL-1 blue cells, and plated on 2% glucose, 2×TY, 100 μg/ml ampicillin plates. Individual colonies were grown for the production of phage particles as previously described (Odegrip et al Proc. Natl. Acad. Sci USA 101 2806-2810 (2004)). NUNC Maxisorp plates were coated with 100 ng/well of anti-FLAG M2 antibody in PBS overnight at 4° C. ELISA assays were performed with bound phage being incubated for 1 hour in 2% BSA in PBS, +/−Human Thrombin (0.1 unit/ml). The assay was developed with SureBlue TMB peroxidase substrate (Insight Biotechnology, Middlesex, UK) and read at 450 nm.

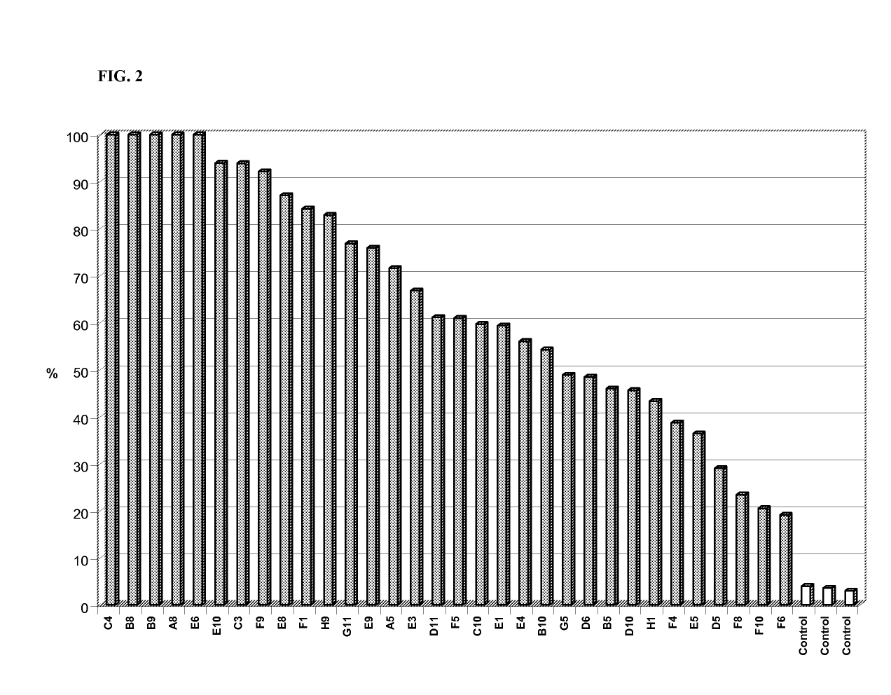

After five rounds of selection a range of resistances to thrombin were found (FIG. 2), with all fifth round selected peptides being more resistant than a control FLAG epitope-thrombin substrate sequence DYKDDDRSGGSGLGPRSG (SEQ ID NO: 002) which has no anti-FLAG binding after one hours incubation in thrombin. Sequence analysis demonstrated that nearly all peptides (70%) had retained the thrombin cleavage site (SEQ ID NO: 003-SEQ ID NO: 027) indicating that the library member peptide is protecting the thrombin substrate peptide from cleavage.

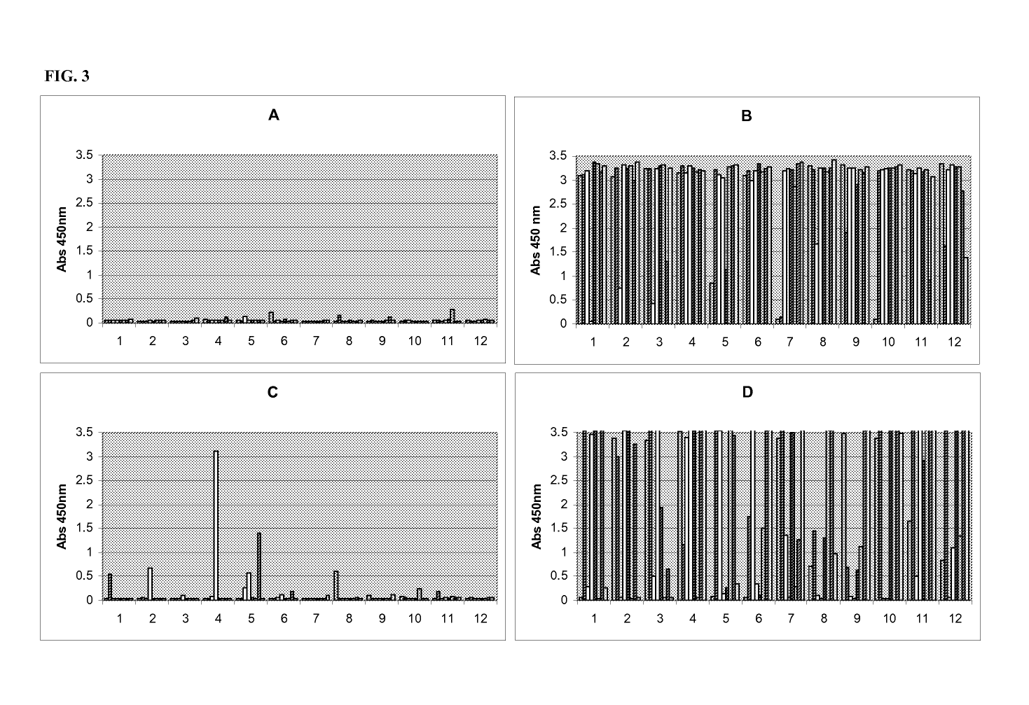

To investigate the resistance to incidental proteases, 94 clones from either unselected library or after five rounds of thrombin resistance selection were incubated with trypsin-agarose for two hours at 25° C. and then assayed by ELISA against anti-FLAG antibody (the antibody is trypsin sensitive, hence the separate incubation steps). Trypsin cleaves after R or after K, thus there are two trypsin sites fixed in the FLAG epitope and thrombin substrate sequence. Even partial resistance to trypsin is surprising as no selection for trypsin resistance was performed and the FLAG epitope is susceptible to trypsin cleavage. Such resistance is observed in round five peptides selected for thrombin resistance, but not in the un-selected library peptides (FIG. 3). Surprisingly, certain of the peptide stabilizer components are capable of protecting bioactive peptides from cleavage by incidental proteases (see Table 1).

### Example 2

### Demonstration of Enrichment of Peptide Stabilizers that Protect Against Cleavage by Chymotrypsin and Trypsin

To further exemplify the invention, protease resistant peptides were selected from a mixed length N-terminal cis display library genetically fused to RepA, under the control of a tac promoter constructed to contain:

a. a FLAG peptide moiety capable of binding to an anti-FLAG antibody (bioactive peptide 1) and,

b. a mixed library containing either twelve, nine and six random amino acids from which protease resistant peptide stabilizer moieties may be selected and,

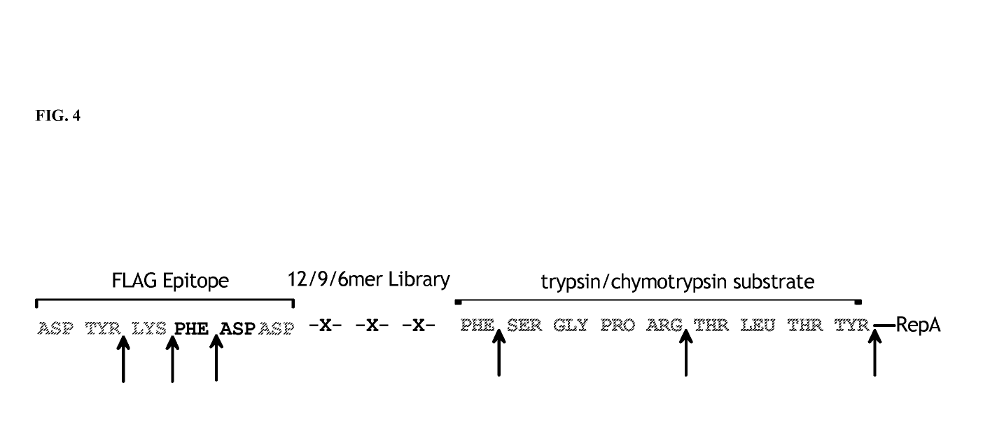

c. a nine amino acid moiety capable of being cleaved by a number of proteases including trypsin and chymotrypsin (bioactive peptide 2) as shown in FIG. 4.

Library construction and in vitro transcription and translation were carried out as described by Odegrip et al (Proc. Natl. Acad. Sci USA 101 2806-2810 (2004). The tac-FLAG-NNB-protease cleavage site-RepA-CIS-ori PCR constructs were prepared by appending a FLAG epitope and either a 12-mer, 9-mer or 6-mer NNB library (where N is any nucleotide and B is either C, T or G), followed by a protease cleavage sequence (encoding FSGPRTLTY—where “R”=P1 for trypsin and thrombin and “F” and “Y”=P1 for chymotrypsin) to the tac promoter by PCR with primers 309, 310 or 311 (SEQ ID NOS: 034-036) and then ligating each to the RepA-CIS-ori region followed by PCR amplification. Equal quantities of the products from each construction were mixed to generate a library encoding 12, 9 or 6 amino acids within the protease resistant peptide stabilizer moiety. In vitro transcription and translation was performed as in Example 1. These were diluted 1:10 in blocking buffer (2% BSA, 0.1 mg/ml herring sperm DNA, in PBS) followed by the addition of one tenth volume of 10× thrombin digestion buffer (200 mM Tris-HCl pH8.4, 1.5 M NaCl, 25 mM CaCl₂). Selection round 1 expressed 9 μg of linear DNA in 150 μl of S-30 lysate, whilst 5 μg DNA and 100 μl lysate were used in subsequent rounds. Protease digestion was carried out by adding chymotrypsin and trypsin each immobilised on agarose beads (SIGMA) followed by incubation for 2 hours at room temperature with mixing by inversion. In round 1, 0.8 units of chymotrypsin and 1.0 units of trypsin were used, whereas 0.4 and 0.5 units, 0.6 and 0.75 units and 0.8 and 1.0 units were used in rounds 2, 3 and 4, respectively. Proteases were removed by centrifugation and the supernatant subjected to selection using biotinylated anti-FLAG (M2) (SIGMA) antibody and Streptavidin-coated Dynal M280 paramagnetic beads as described in Example 1. Thrombin digestion was omitted from these selections. DNA elution, purification and reassembly for subsequent rounds of selection were essentially as described in Example 1.

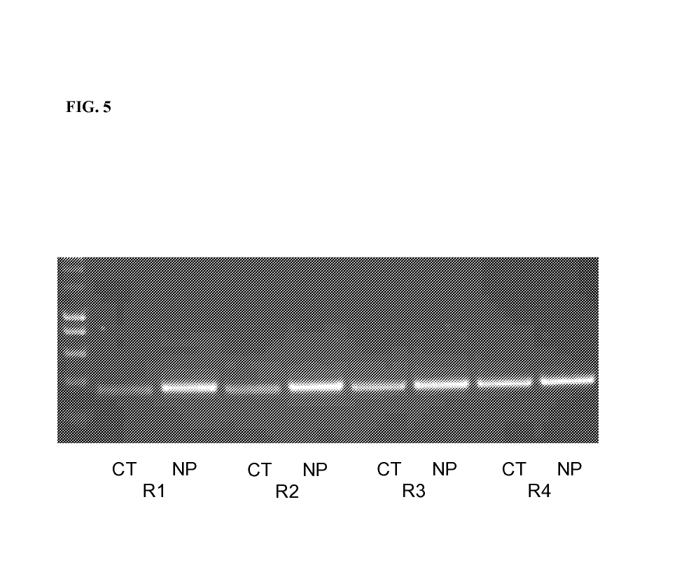

Comparison of the recovery of protease resistant products following expression and protease digestion of equal amounts of library DNA generated after rounds 1-4 demonstrates the selection and enrichment of protease resistant peptide stabilizer moieties. Equal quantities (3.2 μg) of library DNA generated after selection rounds 1-4 were expressed in 100 μl lysate and diluted as described above. Each was divided into two aliquots and 0.4 units of chymotrypsin-agarose and 0.5 units of trypsin-agarose added to one of each aliquot. Following incubation, removal of the proteases and selection of protected peptides using biotinylated anti-FLAG (M2) (SIGMA) antibody and Streptavidin-coated Dynal M280 paramagnetic beads as described above, products recovered by PCR were subjected to agarose gel electrophoresis (FIG. 5). Products were generated in approximately equal quantities from DNA from rounds 1-4 in the absence of protease digestion, indicating no differences in expression, selection and recovery from this material. In the presence of protease digestion, round 1 generates far less product than in its presence. However, in subsequent rounds this difference becomes less marked with the products generated from round 4 material appearing approximately equal both with or without protease digestion. This demonstrates the enrichment of entities within the library capable of stabilizing protease resistance of a bioactive peptide.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

TABLE 1
Peptide Stabilizer Sequences

| SEQID | | 5′-GGCGTACCGATGCGGCCGCTAGACT |
|---|---|---|
| 001 | | AGAACCGCTGCCGGATCGAGGACCVNNVNN |
| | | VNNVNNVNNVNNVNNVNNVNNVNNVNNVNNG |
| | | TGCCAGTAATCATCAAACTTGTAGTC |
| SEQID | Thrombin | DYKDDDRSGGSGLGPRSG |
| 002 | control | |
| | sequence | |
| SEQID | Thrombin | HYPPPSTPYTTD |
| 003 | resistant | |
| SEQID | Thrombin | PTPTNPPQSAAD |
| 004 | resistant | |
| SEQID | Thrombin | ESPRPPARPPND |
| 005 | resistant | |
| SEQID | Thrombin | NHPTTNDGPSVK |
| 006 | resistant | |
| SEQID | Thrombin | PNRNFQQNTNHN |
| 007 | resistant | |
| SEQID | Thrombin | PATNPHTNSNAN |
| 008 | resistant | |
| SEQID | Thrombin | HNVNPNQPHNDT |
| 009 | resistant | |
| SEQID | Thrombin | VPTSTYAITDPT |
| 010 | resistant | |
| SEQID | Thrombin | PTMTRPSNTEAE |
| 011 | resistant | |
| SEQID | Thrombin | PTPSHSTHRDPE |
| 012 | resistant | |
| SEQID | Thrombin | HPHHPSNQAPTD |
| 013 | resistant | |
| SEQID | Thrombin | AAPDETTTPNRD |
| 014 | resistant | |
| SEQID | Thrombin | VNFAATSSNDRD |
| 015 | resistant | |
| SEQID | Thrombin | HDGRPPQHHHPH |
| 016 | resistant | |
| SEQID | Thrombin | MTMGTRPTRDTH |
| 017 | resistant | |
| SEQID | Thrombin | VNKQTTASQAHH |
| 018 | resistant | |
| SEQID | Thrombin | TNFSKDEQPTPD |
| 019 | resistant | |
| SEQID | Thrombin | GRPATAPCTHGN |
| 020 | resistant | |
| SEQID | Thrombin | DRSRASTRRDRH |
| 021 | resistant | |
| SEQID | Thrombin | SRHRTNAGETDH |
| 022 | resistant | |
| SEQID | Thrombin | ATGPIPHTPQGS |
| 023 | resistant | |
| SEQID | Thrombin | TDPPANDNAQPH |
| 024 | resistant | |
| SEQID | Thrombin | RFVSDHNITAAD |
| 025 | resistant | |
| SEQID | Thrombin | QPHNHPRPIKQH |
| 026 | resistant | |
| SEQID | Thrombin | ITPPDNSHTPDE |
| 027 | & Trypsin | |
| | resistant | |
| SEQID | Thrombin | HGHSPSDNANTR |
| 028 | & Trypsin | |
| | resistant | |
| SEQID | Thrombin | HCVHEPQTKHES |
| 029 | & Trypsin | |
| | resistant | |
| SEQID | Thrombin | PSCPNKQDPTHD |
| 030 | & trypsin | |
| | resistant | |
| SEQID | Thrombin | TDCSHNPTDPCE |
| 031 | resistant | |
| SEQID | Thrombin | RAGELGAPADPD |
| 032 | resistant | |
| SEQID | Thrombin | QKPNHDTERELD |
| 033 | resistant | |

1-19. (canceled)

20. A method for identifying peptide stabilizer compounds in vitro, comprising the steps of:
a) expressing a plurality of nucleic acid constructs, wherein each nucleic acid construct encodes a hybrid peptide that comprises:
i) a bioactive moiety; and
ii) a putative peptide stabilizer moiety;
b) exposing the expressed hybrid peptides to one or more proteases;
c) exposing the expressed hybrid peptides to a target molecule which is capable of interacting with the bioactive moiety in a detectable manner; and
d) if a detectable interaction occurs between a target molecule and the bioactive moiety of one or more of the expressed hybrid peptides, identifying said one or more expressed hybrid peptides as comprising a peptide stabilizer compound.

21. A method according to claim 20, further comprising the step of correlating the one or more expressed hybrid peptides of (d) with the corresponding nucleic acid constructs, thereby identifying nucleic acid sequences for the peptide stabilizer compounds.

22. A method according to claim 20, wherein steps (b) and (c) are carried out in the order of (c) then (b), or at the same time

23. A method according to claim 20, wherein the peptide stabilizer moiety comprises an amino acid sequence of between 2 and 20 residues in length.

24. A method according to claim 20, wherein the peptide stabilizer moiety comprises an amino acid sequence of between 2 and 20 residues in length, optionally modified at the N- or C-terminus or both; and its salts, derivatives and functional analogues thereof, wherein said derivatives and functional analogues may comprise naturally occurring or non-natural amino acids, peptide mimetics or peptide analogs.

25. A method according to claim 20, wherein the peptide stabilizer moiety is comprised within the bioactive moiety.

26. A method according to claim 20, wherein the bioactive moiety comprises one or more of the group consisting of: enzymes; hormones; cytokines; antibodies; antibody fragments; analgesics; antipyretics; anti-inflammatory agents; antibiotics; antiviral agents; anti-fungal drugs; cardiovascular drugs; drugs that affect renal function and electrolyte metabolism; drugs that act on the central nervous system; and chemotherapeutic drugs.

27. A peptide stabilizer compound identified by the method of claim 20.

28. A peptide stabilizer compound selected from the group consisting of HYPPPSTPYTTD (SEQ ID NO: 003); PTPTNPPQSAAD (SEQ ID NO: 004); ESPRPPARPPND (SEQ ID NO: 005); NHPTTNDGPSVK (SEQ ID NO: 006); PNRNFQQNTNHN (SEQ ID NO: 007); PATNPHTNSNAN (SEQ ID NO: 008); HNVNPNQPHNDT (SEQ ID NO: 009); VPTSTYAITDPT (SEQ ID NO: 010); PTMTRPSNTEAE (SEQ ID NO: 011); PTPSHSTHRDPE (SEQ ID NO: 012); HPHHPSNQAPTD (SEQ ID NO: 013); AAPDETTTPNRD (SEQ ID NO: 014); VNFAATSSNDRD (SEQ ID NO: 015); HDGRPPQHHHPH (SEQ ID NO: 016); MTMGTRPTRDTH (SEQ ID NO: 017); VNKQTTASQAHH (SEQ ID NO: 018); TNFSKDEQPTPD (SEQ ID NO: 019); GRPATAPCTHGN (SEQ ID NO: 020); DRSRASTRRDRH (SEQ ID NO: 021); SRHRTNAGETDH (SEQ ID NO: 022); ATGPIPHTPQGS (SEQ ID NO: 023); TDPPANDNAQPH (SEQ ID NO: 024); RFVSDHNITAAD (SEQ ID NO: 025); QPHNHPRPIKQH (SEQ ID NO: 026); ITPPDNSHTPDE (SEQ ID NO: 027); HGHSPSDNANTR (SEQ ID NO: 028); HCVHEPQTKHES (SEQ ID NO: 029); PSCPNKQDPTHD (SEQ ID NO: 030); TDCSHNPTDPCE (SEQ ID NO: 031); RAGELGAPADPD (SEQ ID NO: 032); and QKPNHDTERELD (SEQ ID NO: 033).

29. The peptide stabilizer compound of claim 28, which is optionally modified at the N- or C-terminus or both; and its salts, derivatives and functional analogues thereof, wherein said derivatives and functional analogues may comprise naturally occurring or non-natural amino acids, peptide mimetics or peptide analogs.

30. A biologically active peptide comprising a bioactive moiety and a peptide stabilizer compound selected from one or more of the peptide stabilizer compounds set out in claim 28.

31. A biologically active peptide according to claim 30, wherein the biologically active moiety is selected from the group consisting of: enzymes; hormones; cytokines; antibodies; antibody fragments; analgesics; antipyretics; anti-inflammatory agents; antibiotics; antiviral agents; anti-fungal drugs; cardiovascular drugs; drugs that affect renal function and electrolyte metabolism; drugs that act on the central nervous system; and chemotherapeutic drugs.

32. A pharmaceutical composition comprising at least one peptide stabilizer compound identified according to the method of claim 20, a bioactive molecule and a suitable carrier.

33. A pharmaceutical composition according to claim 32, wherein the peptide stabilizer compound is selected from the group consisting of: HYPPPSTPYTTD (SEQ ID NO: 003); PTPTNPPQSAAD (SEQ ID NO: 004); ESPRPPARPPND (SEQ ID NO: 005); NHPTTNDGPSVK (SEQ ID NO: 006); PNRNFQQNTNHN (SEQ ID NO: 007); PATNPHTNSNAN (SEQ ID NO: 008); HNVNPNQPHNDT (SEQ ID NO: 009); VPTSTYAITDPT (SEQ ID NO: 010); PTMTRPSNTEAE (SEQ ID NO: 011); PTPSHSTHRDPE (SEQ ID NO: 012); HPHHPSNQAPTD (SEQ ID NO: 013); AAPDETTTPNRD (SEQ ID NO: 014); VNFAATSSNDRD (SEQ ID NO: 015); HDGRPPQHHHPH (SEQ ID NO: 016); MTMGTRPTRDTH (SEQ ID NO: 017); VNKQTTASQAHH (SEQ ID NO: 018); TNFSKDEQPTPD (SEQ ID NO: 019); GRPATAPCTHGN (SEQ ID NO: 020); DRSRASTRRDRH (SEQ ID NO: 021); SRHRTNAGETDH (SEQ ID NO: 022); ATGPIPHTPQGS (SEQ ID NO: 023); TDPPANDNAQPH (SEQ ID NO: 024); RFVSDHNITAAD (SEQ ID NO: 025); QPHNHPRPIKQH (SEQ ID NO: 026); ITPPDNSHTPDE (SEQ ID NO: 027); HGHSPSDNANTR (SEQ ID NO: 028); HCVHEPQTKHES (SEQ ID NO: 029); PSCPNKQDPTHD (SEQ ID NO: 030); TDCSHNPTDPCE (SEQ ID NO: 031); RAGELGAPADPD (SEQ ID NO: 032); and QKPNHDTERELD (SEQ ID NO: 033).

34. A pharmaceutical composition according to claim 32, wherein the peptide stabilizer compound is selected from the group consisting of: HYPPPSTPYTTD (SEQ ID NO: 003); PTPTNPPQSAAD (SEQ ID NO: 004); ESPRPPARPPND (SEQ ID NO: 005); NHPTTNDGPSVK (SEQ ID NO: 006); PNRNFQQNTNHN (SEQ ID NO: 007); PATNPHTNSNAN (SEQ ID NO: 008); HNVNPNQPHNDT (SEQ ID NO: 009); VPTSTYAITDPT (SEQ ID NO: 010); PTMTRPSNTEAE (SEQ ID NO: 011); PTPSHSTHRDPE (SEQ ID NO: 012); HPHHPSNQAPTD (SEQ ID NO: 013); AAPDETTTPNRD (SEQ ID NO: 014); VNFAATSSNDRD (SEQ ID NO: 015); HDGRPPQHHHPH (SEQ ID NO: 016); MTMGTRPTRDTH (SEQ ID NO: 017); VNKQTTASQAHH (SEQ ID NO: 018); TNFSKDEQPTPD (SEQ ID NO: 019); GRPATAPCTHGN (SEQ ID NO: 020); DRSRASTRRDRH (SEQ ID NO: 021); SRHRTNAGETDH (SEQ ID NO: 022); ATGPIPHTPQGS (SEQ ID NO: 023); TDPPANDNAQPH (SEQ ID NO: 024); RFVSDHNITAAD (SEQ ID NO: 025); QPHNHPRPIKQH (SEQ ID NO: 026); ITPPDNSHTPDE (SEQ ID NO: 027); HGHSPSDNANTR (SEQ ID NO: 028); HCVHEPQTKHES (SEQ ID NO: 029); PSCPNKQDPTHD (SEQ ID NO: 030); TDCSHNPTDPCE (SEQ ID NO: 031); RAGELGAPADPD (SEQ ID NO: 032); and QKPNHDTERELD (SEQ ID NO: 033), wherein said peptide stabilizer is optionally modified at the N- or C-terminus or both; and salts, derivatives and functional analogues thereof, wherein said derivatives and functional analogues may comprise naturally occurring or non-natural amino acids, peptide mimetics or peptide analogs.

35. A pharmaceutical composition according to claim 32, wherein the bioactive molecule is selected from the group consisting of: enzymes; hormones; cytokines; antibodies; antibody fragments; analgesics; antipyretics; anti-inflammatory agents; antibiotics; antiviral agents; anti-fungal drugs; cardiovascular drugs; drugs that affect renal function and electrolyte metabolism; drugs that act on the central nervous system; and chemotherapeutic drugs.

36. A pharmaceutical composition according to claim 32, wherein the peptide stabilizer is attached to the bioactive molecule.

37. A pharmaceutical composition according to claim 32, wherein the peptide stabilizer is comprised within the bioactive molecule.

38. A pharmaceutical composition according to claim 32, wherein the composition is suitable for oral administration.

39. A method for imparting proteolysis resistance to a bioactive peptide molecule comprising, linking a peptide stabilizer compound identified by the method of claim 20 to the bioactive peptide molecule.

40. A method according to claim 39, wherein the peptide stabilizer compound is conjugated to the bioactive molecule.

41. A method according to claim 39, wherein the peptide stabilizer compound is genetically fused to the bioactive molecule.


SEQUENCE LISTING (39 sequences)

SEQ ID NO: 1 - DNA, 112 bp, Artificial Sequence
  Description of Artificial Sequence Synthetic polynucleotide
ggcgtaccgatgcggccgctagactagaaccgctgccggatcgaggaccvnnvnnvnnvnnvnnvnnvnnvnnvnnvnnvnnvnngtgccagtaatcatcaaacttgtagtc

SEQ ID NO: 2 - protein, 18 aa, Artificial Sequence
  Description of Artificial Sequence Synthetic thrombin control peptide
DYKDDDRSGGSGLGPRSG

SEQ ID NO: 3 - protein, 12 aa, Artificial Sequence
  Description of Artificial Sequence Synthetic thrombin resistant peptide
HYPPPSTPYTTD

SEQ ID NO: 4 - protein, 12 aa, Artificial Sequence
  Description of Artificial Sequence Synthetic thrombin resistant peptide
PTPTNPPQSAAD

SEQ ID NO: 5 - protein, 12 aa, Artificial Sequence
  Description of Artificial Sequence Synthetic thrombin resistant peptide
ESPRPPARPPND

SEQ ID NO: 6 - protein, 12 aa, Artificial Sequence
  Description of Artificial Sequence Synthetic thrombin resistant peptide
NHPTTNDGPSVK

SEQ ID NO: 7 - protein, 12 aa, Artificial Sequence
  Description of Artificial Sequence Synthetic thrombin resistant peptide
PNRNFQQNTNHN

SEQ ID NO: 8 - protein, 12 aa, Artificial Sequence
  Description of Artificial Sequence Synthetic thrombin resistant peptide
PATNPHTNSNAN

SEQ ID NO: 9 - protein, 12 aa, Artificial Sequence
  Description of Artificial Sequence Synthetic thrombin resistant peptide
HNVNPNQPHNDT

SEQ ID NO: 10 - protein, 12 aa, Artificial Sequence
  Description of Artificial Sequence Synthetic thrombin resistant peptide
VPTSTYAITDPT

SEQ ID NO: 11 - protein, 12 aa, Artificial Sequence
  Description of Artificial Sequence Synthetic thrombin resistant peptide
PTMTRPSNTEAE

SEQ ID NO: 12 - protein, 12 aa, Artificial Sequence
  Description of Artificial Sequence Synthetic thrombin resistant peptide
PTPSHSTHRDPE

SEQ ID NO: 13 - protein, 12 aa, Artificial Sequence
  Description of Artificial Sequence Synthetic thrombin resistant peptide
HPHHPSNQAPTD

SEQ ID NO: 14 - protein, 12 aa, Artificial Sequence
  Description of Artificial Sequence Synthetic thrombin resistant peptide
AAPDETTTPNRD

SEQ ID NO: 15 - protein, 12 aa, Artificial Sequence
  Description of Artificial Sequence Synthetic thrombin resistant peptide
VNFAATSSNDRD

SEQ ID NO: 16 - protein, 12 aa, Artificial Sequence
  Description of Artificial Sequence Synthetic thrombin resistant peptide
HDGRPPQHHHPH

SEQ ID NO: 17 - protein, 12 aa, Artificial Sequence
  Description of Artificial Sequence Synthetic thrombin resistant peptide
MTMGTRPTRDTH

SEQ ID NO: 18 - protein, 12 aa, Artificial Sequence
  Description of Artificial Sequence Synthetic thrombin resistant peptide
VNKQTTASQAHH

SEQ ID NO: 19 - protein, 12 aa, Artificial Sequence
  Description of Artificial Sequence Synthetic thrombin resistant peptide
TNFSKDEQPTPD

SEQ ID NO: 20 - protein, 12 aa, Artificial Sequence
  Description of Artificial Sequence Synthetic thrombin resistant peptide
GRPATAPCTHGN

SEQ ID NO: 21 - protein, 12 aa, Artificial Sequence
  Description of Artificial Sequence Synthetic thrombin resistant peptide
DRSRASTRRDRH

SEQ ID NO: 22 - protein, 12 aa, Artificial Sequence
  Description of Artificial Sequence Synthetic thrombin resistant peptide
SRHRTNAGETDH

SEQ ID NO: 23 - protein, 12 aa, Artificial Sequence
  Description of Artificial Sequence Synthetic thrombin resistant peptide
ATGPIPHTPQGS

SEQ ID NO: 24 - protein, 12 aa, Artificial Sequence
  Description of Artificial Sequence Synthetic thrombin resistant peptide
TDPPANDNAQPH

SEQ ID NO: 25 - protein, 12 aa, Artificial Sequence
  Description of Artificial Sequence Synthetic thrombin resistant peptide
RFVSDHNITAAD

SEQ ID NO: 26 - protein, 12 aa, Artificial Sequence
  Description of Artificial Sequence Synthetic thrombin resistant peptide
QPHNHPRPIKQH

SEQ ID NO: 27 - protein, 12 aa, Artificial Sequence
  Description of Artificial Sequence Synthetic thrombin and trypsin resistant peptide
ITPPDNSHTPDE

SEQ ID NO: 28 - protein, 12 aa, Artificial Sequence
  Description of Artificial Sequence Synthetic thrombin and trypsin resistant peptide
HGHSPSDNANTR

SEQ ID NO: 29 - protein, 12 aa, Artificial Sequence
  Description of Artificial Sequence Synthetic thrombin and trypsin resistant peptide
HCVHEPQTKHES

SEQ ID NO: 30 - protein, 12 aa, Artificial Sequence
  Description of Artificial Sequence Synthetic thrombin and trypsin resistant peptide
PSCPNKQDPTHD

SEQ ID NO: 31 - protein, 12 aa, Artificial Sequence
  Description of Artificial Sequence Synthetic thrombin resistant peptide
TDCSHNPTDPCE

SEQ ID NO: 32 - protein, 12 aa, Artificial Sequence
  Description of Artificial Sequence Synthetic thrombin resistant peptide
RAGELGAPADPD

SEQ ID NO: 33 - protein, 12 aa, Artificial Sequence
  Description of Artificial Sequence Synthetic thrombin resistant peptide
QKPNHDTERELD

SEQ ID NO: 34 - DNA, 125 bp, Artificial Sequence
  Description of Artificial Sequence Synthetic primer
cgtaccgatgcggccgctagcagatcagaaccgctgccgtaggtcagggtacgcggaccagaaaavnnvnnvnnvnnvnnvnngtaatcatcaaacttgtagtcggccatggtagatcctgtttc

SEQ ID NO: 35 - DNA, 134 bp, Artificial Sequence
  Description of Artificial Sequence Synthetic primer
cgtaccgatgcggccgctagcagatcagaaccgctgccgtaggtcagggtacgcggaccagaaaavnnvnnvnnvnnvnnvnnvnnvnnvnngtaatcatcaaacttgtagtcggccatggtagatcctgtttc

SEQ ID NO: 36 - DNA, 143 bp, Artificial Sequence
  Description of Artificial Sequence Synthetic primer
cgtaccgatgcggccgctagcagatcagaaccgctgccgtaggtcagggtacgcggaccagaaaavnnvnnvnnvnnvnnvnnvnnvnnvnnvnnvnnvnngtaatcatcaaacttgtagtcggccatggtagatcctgtttc

SEQ ID NO: 37 - protein, 9 aa, Artificial Sequence
  Description of Artificial Sequence Synthetic peptide
FSGPRTLTY

SEQ ID NO: 38 - protein, 23 aa, Artificial Sequence
  Description of Artificial Sequence Synthetic construct
DYKFDDXXXXXXXXXXXXGPRSG

SEQ ID NO: 39 - protein, 27 aa, Artificial Sequence
  Description of Artificial Sequence Synthetic construct
DYKFDDXXXXXXXXXXXXFSGPRTLTY